(19) United States
(12) Patent Application Publication
(10) Pub. No.: US 2013/0210942 A1
(43) Pub. Date: Aug. 15, 2013

(54) MODIFIED FISCHER-TROPSCH MONOLITH CATALYSTS AND METHODS FOR PREPARATION AND USE THEREOF

(71) Applicants: Charles Leonard Kibby, Benicia, CA (US); Robert James Saxton, JR., Pleasanton, CA (US); Kandaswamy Jothimurugesan, Hercules, CA (US); Tapan Kumar Das, Albany, CA (US); Howard Steven Lacheen, Richmond, CA (US); Michael Bartz, (US); Alfred Has, (US)

(72) Inventors: Charles Leonard Kibby, Benicia, CA (US); Robert James Saxton, JR., Pleasanton, CA (US); Kandaswamy Jothimurugesan, Hercules, CA (US); Tapan Kumar Das, Albany, CA (US); Howard Steven Lacheen, Richmond, CA (US); Michael Bartz, (US); Alfred Has, (US)

(73) Assignee: Chevron U.S.A Inc., San Ramon, CA (US)

(21) Appl. No.: 13/396,466

(22) Filed: Feb. 14, 2012

(51) Int. Cl. B01J 35/02; B01J 23/75; C07C 1/04; B01J 37/02

(52) U.S. Cl. 518/728; 502/100; 502/300; 502/325

(57) ABSTRACT

Disclosed are hybrid synthesis gas conversion catalysts containing at least one Fischer-Tropsch component and at least one acidic component deposited on a monolith catalyst support for use in synthesis gas conversion processes and methods for preparing the catalysts. Also disclosed are synthesis gas conversion processes in which the hybrid synthesis gas conversion catalysts are contacted with synthesis gas to produce a hydrocarbon product containing at least 50 wt % C₅₊ hydrocarbons. Also disclosed are synthesis gas conversion processes in which at least one layer of Fischer-Tropsch component deposited onto a monolith support is alternated with at least one layer of acidic component in a fixed bed reactor.

### FIELD

The disclosure relates to synthesis gas conversion catalysts deposited on monolith catalyst supports for use in synthesis gas conversion processes and to methods for preparing the catalysts. The disclosure further relates to processes using the catalysts for converting synthesis gas to liquid hydrocarbon products such as fuels.

### BACKGROUND

High quality fuels remain in high demand. Fischer-Tropsch synthesis, which involves the production of hydrocarbons by the catalyzed reaction of mixtures of carbon monoxide (CO) and hydrogen (H₂), also referred to as synthesis gas or syngas, can convert carbon-based materials, such as natural gas, into liquid fuels and high-value chemicals. Fischer-Tropsch synthesis is one of the more attractive, direct and environmentally acceptable paths to high quality transportation fuels derived from natural gas. The Fischer-Tropsch process can produce a wide variety of materials depending on catalyst and process conditions. Fischer-Tropsch catalysts are based on group VIII metals such as, for example, iron, cobalt, nickel and ruthenium. For example, cobalt and ruthenium make primarily paraffinic products, cobalt tending towards a heavier product slate, e.g., containing C₂₀₊, while ruthenium tends to produce more distillate type paraffins, e.g., C₅-C₂₀₊, depending on conditions. Processes using such catalyst are generally governed by the Anderson-Schulz-Flory (ASF) polymerization kinetics. In general the product distribution of hydrocarbons formed during the Fischer-Tropsch process can be expressed as:

Wₙ/n=(1−α)²αⁿ⁻¹

where Wₙ is the weight fraction of hydrocarbon molecules containing n carbon atoms, and α is the chain growth probability for a given catalyst and process conditions.

Known commercial processes for converting synthesis gas to liquid hydrocarbon products utilizing Fischer-Tropsch processes produce an effluent which contains a solid wax fraction along with C₁₋₄ light gas, CO₂, C₅₊ liquid products and water. Upon leaving the reactor, the wax fraction must be separated from the effluent before the light gases, liquid products and water can be separated from one another and further processed. In order to remove the wax fraction, a heated separation step is generally necessary in order to keep the wax in a liquid state. In some cases, significant amounts of wax must be removed from the effluent, i.e. roughly 30-35% by weight. In slurry bed operations, wax must be separated from the catalyst, following which the wax is filtered.

It is known that Fischer-Tropsch catalysts in slurry and fluid bed operations produce lower levels of methane than Fischer-Tropsch catalysts in fixed bed operations. However, fixed bed reactors have inherent advantages in terms of catalyst-product separation.

Hybrid Fischer-Tropsch catalyst systems which further include an acidic component, such as a zeolite, have been developed which are capable of limiting product chain growth in the Fischer-Tropsch reaction to a desired product distribution. Such hybrid Fischer-Tropsch catalysts have been developed for use in fixed bed reactors, typically having a particle size of between about 0.5 mm and about 6 mm. The particle size is large enough to avoid high pressure drops. Such catalysts are generally prepared by depositing a Fischer-Tropsch active metal onto a shaped or extruded oxide support by solution deposition, wetness impregnation or similar technique. During the course of a Fischer-Tropsch reaction, carbon monoxide and hydrogen diffuse into the interior of the catalyst particles. Since hydrogen diffuses more rapidly into and out of the pores of the catalyst particles, hydrogen partial pressure has a tendency to build inside the pores, resulting in a propensity to completely hydrogenate carbon monoxide to methane.

There is a need for processes utilizing a hybrid Fischer-Tropsch catalyst system in fixed bed operations, with its ability to produce a liquid hydrocarbon product having a C₂₁₊ paraffin content of 5% or less with high levels of overall CO conversion, while avoiding excessive formation of methane.

### SUMMARY

Hybrid Fischer-Tropsch monolith catalysts and method for forming hybrid Fischer-Tropsch monolith catalysts are provided. The hybrid Fischer-Tropsch monolith catalysts include at least one catalyst layer including a synthesis gas conversion component and an acidic component deposited on a monolithic support.

According to another embodiment, a process for synthesis gas conversion is provided, the process including contacting a synthesis gas feed comprising hydrogen and carbon monoxide having a H₂/CO ratio between about 1.3 and about 2.0 with a hybrid Fischer-Tropsch monolith catalyst in a reactor at a temperature from about 200° C. to about 260° C., a pressure from about 5 to about 30 atmospheres, and a gaseous hourly space velocity less than 20,000 volumes of gas per volume of catalyst per hour to produce a hydrocarbon product containing at least 50 wt % C₅₊ hydrocarbons.

According to another embodiment, a process for synthesis gas conversion is provided, the process including contacting a synthesis gas feed comprising hydrogen and carbon monoxide having a H₂/CO ratio between about 1.3 and about 2.0 with a Fischer-Tropsch monolith catalyst comprising a monolithic support and a catalyst layer comprising a synthesis gas conversion component deposited on the monolithic support in an alternating arrangement with a catalyst bed comprising acidic component particles in a reactor at a temperature from about 200° C. to about 260° C., a pressure from about 5 to about 40 atmospheres, and a gaseous hourly space velocity less than 20,000 volumes of gas per volume of catalyst per hour to produce a hydrocarbon product containing at least 50 wt % C₅₊ hydrocarbons.

### DESCRIPTION OF THE DRAWINGS

These and other objects, features and advantages of the present invention will become better understood with regard to the following description, appended claims and accompanying drawings where:

FIG. 1 is a perspective view of a monolithic catalyst support according to one exemplary embodiment.

FIGS. 2A-C are top plan views of a hybrid Fischer-Tropsch monolith catalyst according to alternative exemplary embodiments.

FIG. 3 is a simplified cross-sectional view of a reactor including a synthesis gas catalyst supported by a monolithic catalyst support in an alternating arrangement with a catalyst bed of acidic component particles according to one exemplary embodiment.

### DETAILED DESCRIPTION

According to some embodiments, a hybrid Fischer-Tropsch monolith catalyst is provided which includes a catalyst layer comprising at least one synthesis gas conversion component and at least one acidic component deposited on a monolithic support. According to other embodiments, a hybrid Fischer-Tropsch monolith catalyst is provided which includes a plurality of catalyst layers deposited on a monolithic support. The plurality of catalyst layers contain at least one syngas conversion component, also referred to interchangeably as a “Fischer-Tropsch component” or a “Fischer-Tropsch metal,” to be described hereinafter, and at least one acidic component, to be described hereinafter. The Fischer-Tropsch component and the acidic component can be present in the same catalyst layer(s) or in separate catalyst layers.

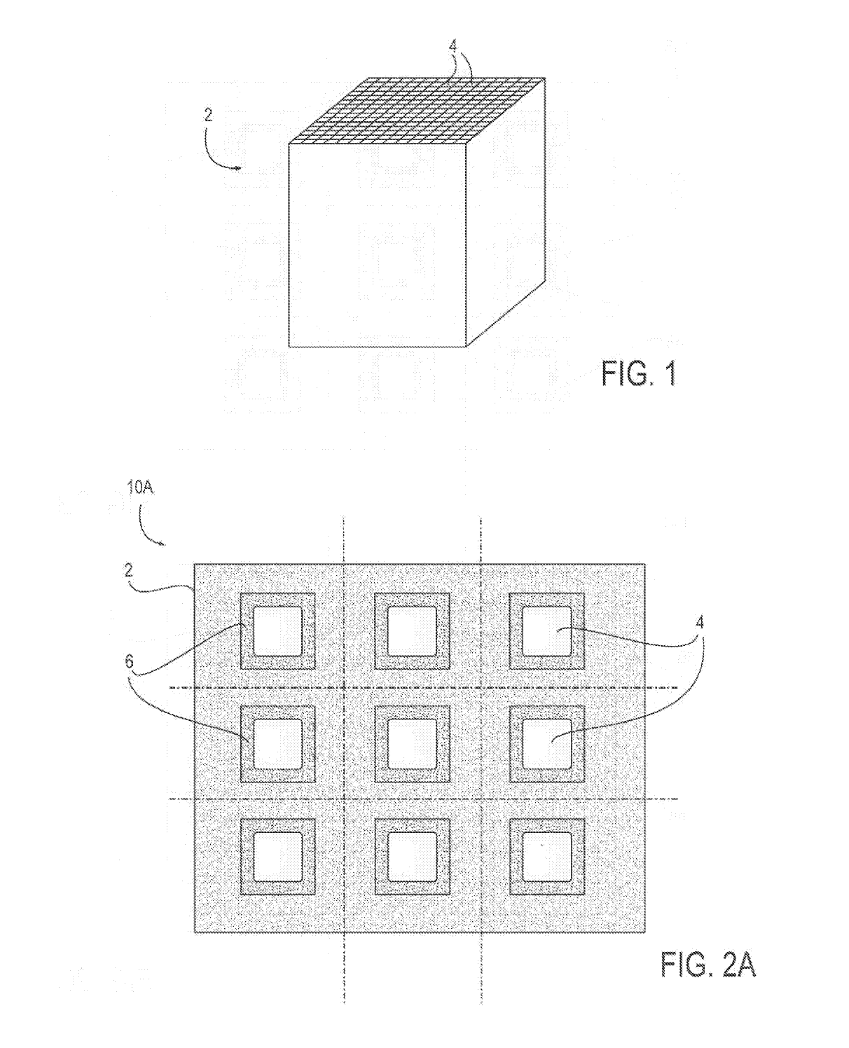

The monolithic support can be any body of material suitable for use as a structural support for a catalyst to be subjected to the conditions of the synthesis gas conversion reaction, to be described hereinafter. Suitable monolithic catalyst supports include, but are not limited to, monoliths formed of ceramic material and monoliths formed of metallic material. Ceramic monoliths can be formed by extrusion. Metallic monoliths can be formed of metallic material such as corrugated metal. The term monolithic support is intended to encompass structural supports including, but not limited to, honeycomb structures, open cell foams, microchannel structures, and blocks of interconnected fibers. Monolith supports which provide for exchange of gases and vapors between internal channels and channels adjacent to the exterior of the monolith support or the reactor wall can provide improved heat removal and temperature control. As an example, FIG. 1 illustrates a monolithic support 10 having a generally honeycomb structure with separate parallel channels 12 having a rectangular cross-section. The channels can be straight as shown, or they may include curvature and/or changes of direction. The channels can have a variety of cross-sectional shapes, including rectangular, square, circular, oval, triangular, etc. Those skilled in the art may identify other support structures and other geometries suitable for use in the monolith catalysts of the present disclosure.

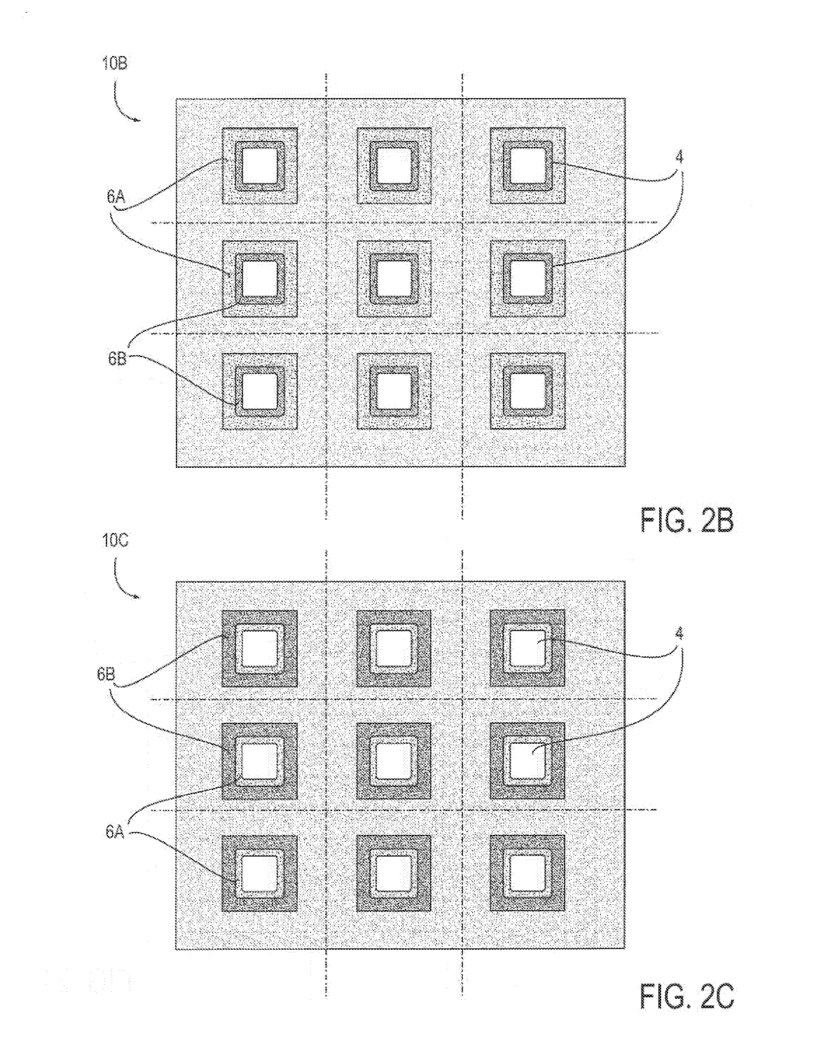

In some embodiments, a catalyst layer deposited on the monolithic support contains both a Fischer-Tropsch component and the acidic component. FIG. 2A is a top plan view of a coated monolithic support 10a in which such a catalyst layer 2 coats the surfaces of the channels 12 of the monolithic support. The catalyst layer can contain between about 10 and about 100 mg, even between about 10 and about 30 mg, of cobalt per gram of monolithic support, depending on the dimensions of the cells of the monolithic support. The catalyst components can be combined as separate, discrete particles of synthesis gas conversion component and acidic component in a slurry including a liquid medium which is then deposited onto the monolithic support.

Alternatively, the catalyst components can be combined in discrete hybrid or integral particles, each particle containing the synthesis gas conversion component and the acidic component. In one embodiment, integral hybrid Fischer-Tropsch catalyst extrudate particles are prepared and then pulverized to a desired particle size, e.g. from about 20 to about 50 μm, and the pulverized catalyst particles are then combined with a liquid medium to form the slurry. The preparation of integral, hybrid Fischer-Tropsch catalyst extrudate particles is known from the disclosure of U.S. Patent Publication No. 2010/0160464 A1, herein incorporated by reference in its entirety.

Alternatively, the Fischer-Tropsch component and the acidic component can be deposited on the monolithic support in separate catalyst layers. In one embodiment, as illustrated in FIG. 2B, a catalyst layer 4 of synthesis gas conversion component, also referred to as the Fischer-Tropsch layer, is deposited onto the surfaces of the channels 12 of the monolithic support, and a catalyst layer 6 of acidic component, also referred to as the zeolite layer, is deposited onto the Fischer-Tropsch layer 4 to form a coated monolithic support 10b.

In another embodiment, as illustrated in FIG. 2C, a zeolite layer 6 is deposited onto the surfaces of the channels 12 of the monolithic support, and a Fischer-Tropsch layer 4 is deposited onto the zeolite layer 6 to form a coated monolithic support 10c. In some embodiments, the Fischer-Tropsch layer can contain from about 30 to about 50 mg of Fischer-Tropsch metal per gram of monolithic support. The zeolite layer can contain from about 12 to about 15 g of acidic component per gram of Fischer-Tropsch metal in the Fischer-Tropsch layer.

For slurry stability, the pH of the slurry is near neutral, i.e., from about 5 to about 8. The slurry can be deposited onto the monolithic support by any suitable means, such as by wash coating, also referred to as dip coating. Wash coating refers to a process in which the monolithic support is dipped into the slurry and removed, thereby coating the monolithic support. The support can be dipped once or multiple times to achieve a desired catalyst layer thickness or a desired catalyst amount. Between coatings, excess slurry can be removed from the monolithic support by any known means, such as by centrifugation, blowing with air or the like, and the coated monolithic support can be dried and or calcined at temperatures between 80° C. and 400° C. Alternatively, the slurry can be deposited onto the monolithic support by spraying, vapor deposition, impregnation or the like.

The thickness of the catalyst layers deposited on the monolithic support can vary from about 50 to about 300 μm, and can be tuned to optimize the effects of the catalyst layer thickness. The thickness of the catalyst layers will in part determine the catalytic activity in the reaction. The thicker the catalyst layer, the more important diffusion effects will be in determining specific activity and selectivity. Additionally, if the catalyst layer is sufficiently thick to impede flow through the channels of the monolith, pressure drop in the reactor may become excessively high. Pressure drop will be determined by the channel dimensions and the cell density of the monolith support. For these reasons, smaller catalyst layer thicknesses may be preferred. However, smaller catalyst layer thicknesses also correspond to lower catalyst loading per unit volume and lower volumetric activities. In general, after the catalyst layers have been deposited, it has been found to be desirable to have a channel opening of at least about 300 μm.

The hybrid Fischer-Tropsch monolith catalyst optionally contains an interfacial layer between layers, such as deposited on the monolithic support between the monolithic support and the catalyst layer, or between multiple catalyst layers, to aid in adhesion of adjacent layers. Similarly, during preparation, the surface of each layer of the hybrid Fischer-Tropsch monolith catalyst can optionally be subjected to a modification to create a rough surface thereby improving the adhesion of subsequently applied, adjacent layers.

In one embodiment, a syngas conversion process is conducted in which the hybrid Fischer-Tropsch monolith catalyst of the present disclosure is contacted with a syngas feed including hydrogen and carbon monoxide having a H₂/CO ratio between about 1.3 and about 2.0 in a reactor at a temperature from about 200° C. to about 260° C., a pressure from about 5 to about 30 atmospheres, and a gaseous hourly space velocity less than 20,000 volumes of gas per volume of catalyst per hour. In general, higher temperature, higher H₂/CO ratio, and lower pressure all favor making lighter products. The process results in a hydrocarbon product containing at least 50 wt % C₅₊ hydrocarbons.

In some embodiments, the resulting hydrocarbon product has a cloud point as determined by ASTM D 2500-09 of about 15° C. or less, even about 10° C. or less, even about 5° C. or less, and even as low as about 2° C. Cloud point refers to the temperature below which wax in a liquid hydrocarbon product forms a cloudy appearance as the wax forms an emulsion with the liquid phase of the product. Cloud point indicates the tendency of the product to plug pumps, filters or small orifices at cold operating temperatures. Note that a 6° C. cloud point is typical for a Number 2 diesel. In some embodiments, the liquid hydrocarbon product is substantially free of solid wax. By “substantially free of solid wax” is meant that the product is a single liquid phase at ambient conditions without the presence of a visible solid wax phase, and containing no greater than 5 wt % C₂₁₊ normal paraffins. In such case, the liquid hydrocarbon product need not be further hydrocracked or hydroisomerized in order to arrive at a wax free product composition.

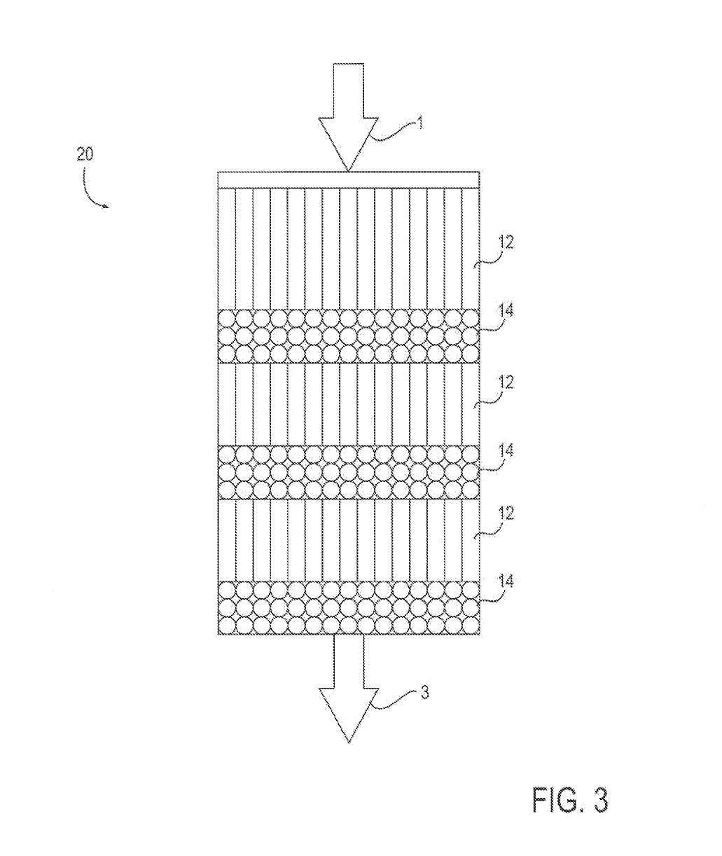

In one embodiment, a Fischer-Tropsch monolith catalyst can be prepared with a Fischer-Tropsch component in the catalyst layer deposited on the monolithic support without the acidic component. A reactor can be loaded with at least one layer of the Fischer-Tropsch monolith catalyst in an alternating arrangement with at least one layer of a separate catalyst bed of acidic component. Referring to FIG. 3, a fixed bed reactor 20 is provided with alternating layers of Fischer-Tropsch monolith catalyst 12 and catalyst bed of zeolite 14. The number of alternating layers shown is for illustration only; the number of layers can vary. In this embodiment, a synthesis gas feed 1 comprising hydrogen and carbon monoxide having a H₂/CO ratio from about 1.3 to about 2.0 can be contacted with the Fischer-Tropsch monolith catalyst 12 in alternating arrangement with the bed of zeolite 14 at a temperature from about 200° C. to about 260° C., even from about 225° C. to about 260° C., a pressure from about 5 to about 40 atmospheres, and a gaseous hourly space velocity less than 20,000 volumes of gas per volume of catalyst per hour. The resulting hydrocarbon product 3 can contain at least 50 wt % C₅+ hydrocarbons.

Throughout the present disclosure, the syngas conversion or Fischer-Tropsch component of the catalyst includes a Group VIII of the Periodic Table metal component, preferably cobalt, iron and/or ruthenium. References to the Periodic Table and groups thereof used herein refer to the IUPAC version of the Periodic Table of Elements described in the 68th Edition of the Handbook of Chemistry and Physics (CPC Press). The catalyst further includes a catalyst carrier or support. The catalyst carrier is preferably porous, such as a porous inorganic refractory oxide, preferably alumina, silica, titania, zirconia or combinations thereof. The optimum amount of catalytically active metal present on the carrier depends inter alia on the specific catalytically active metal. Typically, the amount of cobalt present in the catalyst may range from 1 to 100 parts by weight per 100 parts by weight of carrier material, preferably from 10 to 50 parts by weight per 100 parts by weight of carrier material.

The catalytically active Fischer-Tropsch component may be present in the catalyst together with one or more metal promoters or co-catalysts. The promoters may be present as metals or as metal oxide, depending upon the particular promoter concerned. Suitable promoters include metals or oxides of metals from Groups IA, IB, IVB, VB, VIIB and/or VIIB of the Periodic Table, lanthanides and/or the actinides or oxides of the lanthanides and/or the actinides. As an alternative or in addition to the metal oxide promoter, the catalyst may comprise a metal promoter selected from Groups VIIB and/or VIII of the Periodic Table.

The acidic component of the catalyst can be an acid catalyst material such as amorphous silica-alumina or tungstated zirconia or a zeolitic or non-zeolitic crystalline molecular sieve. Examples of suitable molecular sieves include zeolite Y, zeolite X and the so called “ultra stable zeolite Y” and high structural silica:alumina ratio zeolite Y such as for example described in U.S. Pat. Nos. 4,401,556, 4,820,402 and 5,059,567, herein incorporated by reference. Small crystal size zeolite Y, such as described in U.S. Pat. No. 5,073,530, herein incorporated by reference, can also be used. Other zeolites which show utility include those designated as SSZ-13, SSZ-33, SSZ-46, SSZ-53, SSZ-55, SSZ-57, SSZ-58, SSZ-59, SSZ-64, ZSM-5, ZSM-11, ZSM-12, ZSM-23, H-Y, beta, mordenite, SSZ-74, ZSM-48, TON type zeolites, ferrierite, SSZ-60 and SSZ-70. Non-zeolitic molecular sieves which can be used include, for example silicoaluminophosphates (SAPO), ferroaluminophosphate, titanium aluminophosphate and the various ELAPO molecular sieves described in U.S. Pat. No. 4,913,799 and the references cited therein. Details regarding the preparation of various non-zeolite molecular sieves can be found in U.S. Pat. No. 5,114,563 (SAPO); U.S. Pat. No. 4,913,799 and the various references cited in U.S. Pat. No. 4,913,799, hereby incorporated by reference in their entirety. Mesoporous molecular sieves can also be included, for example the M41S family of materials (J. Am. Chem. Soc. 1992, 114, 10834-10843), MCM-41 (U.S. Pat. Nos. 5,246,689, 5,198,203, 5,334,368), and MCM48 (Kresge et al., Nature 359 (1992) 710).

The amount of acidic component used in the catalyst can be suitably varied to obtain the desired product. For instance, if the amount of acidic component is too low, there may be insufficient cracking to remove a desired amount of wax; whereas if too much acidic component is used, there may be excessive cracking and the resulting product may be lighter than desired.

In one embodiment, the catalyst comprises synthesis gas conversion component and acidic component disposed on integral particles such as catalysts described in U.S. Patent Publication No. 2010/0160464 A1, herein incorporated by reference in its entirety.

The use of hybrid Fischer-Tropsch monolith catalysts as described herein has a number of advantages over the use of hybrid Fischer-Tropsch fixed bed catalysts in pellet, powder or extruded form. For one, processes and systems using hybrid Fischer-Tropsch monolith catalysts have lower pressure drops, which in turn allows for construction of longer reactor tubes and consequently fewer reactor tubes. For another, heat from the exothermic Fischer-Tropsch reaction can be more readily removed, depending on the geometry of the channels of the monolithic support as well as the monolithic support material. In particular, metallic monolithic support materials which allow exchange of gases and vapors between innermost channels and outermost channels allow heat to be readily removed.

Another advantage of processes and systems using hybrid Fischer-Tropsch monolith catalysts is lower production of methane by such processes and systems. Without wishing to be bound by theory, it is believed that the lower production of methane is a result of the smaller particle size used in the catalyst layers on the hybrid Fischer-Tropsch monolith catalysts with their significantly shorter diffusion paths than fixed bed catalysts in pellet, powder or extruded form. The average layer thickness of the deposited hybrid Fischer-Tropsch or conventional Fischer-Tropsch catalyst on a monolith support can be less than about 300 μm in diameter. Compared to an extrudate particle having a diameter of at least 1 mm, the diffusion path in the monolith catalysts should be much shorter which means less diffusion resistance difference between CO and the smaller H₂ which should result, in theory, in lower methane. CO hydrogenation is a function of H₂ concentration so when H₂/CO ratio is much greater than 2, as might be the case in a large particle, these conditions favor methane formation. Conventional forms of hybrid Fischer-Tropsch catalysts for fixed bed operations have a higher selectivity to methane due to higher concentration of methane inside the catalyst particles. Note that, as is known to those skilled in the art, the formation mechanism for methane is partly independent of Fischer-Tropsch synthesis.

### EXAMPLES

### Example 1

A hybrid Fischer-Tropsch catalyst of composition 7.5% Co/0.19% Ru/ZSM-12/Al₂O₃ was prepared as described in U.S. Pat. No. 7,943,674, herein incorporated by reference in its entirety. The catalyst was milled in a small colloid mill to particles having a particle size of about 25 μm. The powder was suspended in water to a solids content of 30 wt %. The pH of the suspension was about 6 for the catalysts tested.

From a commercial cordierite monolith cylinder available from Corning Inc., Corning, N.Y., was cut a 225 cells per inch sample of approximately 50 mm in length. The hybrid Fischer-Tropsch catalyst was deposited onto the monolith sample via a series of sequential dip coatings with intermediate drying and calcination at 300° C. Excess liquid was removed by centrifugation. Approximately 20 mg cobalt/gram monolith support was coated on the monolith sample with the dimensions and characteristics aforementioned. The monolith sample was crushed and sieved to approximately 1 mm size fragments and diluted with alumina.

The catalyst-coated fragments were then placed in a 6 mm cylindrical reactor tube. A catalyst prepared as described above was subjected to activation and a Fischer-Tropsch synthesis run as described in U.S. Pat. No. 7,943,674. Results are given in Table 1.

### Example 2

A Fischer-Tropsch catalyst of composition 20% Co/0.5% Ru/4.2% Mn/Al₂O₃ was prepared as described in U.S. Pat. No. 4,585,798, herein incorporated by reference in its entirety. ZSM-12 catalyst in the acid form with a silica/alumina ratio of 90 was obtained from Zeolyst. The two catalysts were milled separately in a small colloid mill. The Fischer-Tropsch catalyst was milled to particles having a size of approximately 5 μm. The ZSM-12 powder was milled to particles having a size of approximately 25 μm. The powders were suspended in water in a ratio of zeolite catalyst to Fischer-Tropsch catalyst of from about 2:1 to about 2.5:1 wt/wt to a total solids content of 30 wt %. The pH of the suspension was about 6 for the catalysts tested.

From a commercial cordierite monolith cylinder available from Corning Inc., Corning, N.Y., was cut a 225 cells per inch sample of approximately 50 mm in length. The hybrid Fischer-Tropsch catalyst was deposited onto the monolith sample via a series of sequential dip coatings with intermediate drying and calcination at 300° C. Excess liquid was removed by centrifugation. Approximately 20 mg cobalt/gram monolith support was coated on the monolith sample with the dimensions and characteristics aforementioned. The monolith sample was crushed and sieved to approximately 1 mm size fragments and diluted with alumina.

The catalyst-coated fragments were then placed in a 6 mm cylindrical reactor tube. A catalyst prepared as described above was subjected to activation and a Fischer-Tropsch synthesis run as described in U.S. Pat. No. 7,943,674. Results are given in Table 1.

### Comparative Example

A Fischer-Tropsch catalyst of composition 20% Co/0.5% Ru/4.2% Mn/Al₂O₃ was prepared as described in U.S. Pat. No. 4,585,798, herein incorporated by reference in its entirety. The Fischer-Tropsch catalyst was milled in a small colloid mill to particles having a size of approximately 5 μm. The particles were suspended in water at a total solids content of 30 wt %. The pH of the suspension was about 6.

From a commercial cordierite monolith cylinder available from Corning Inc., Corning, N.Y., was cut a 225 cells per inch sample of approximately 50 mm in length. The Fischer-Tropsch catalyst was deposited onto the monolith sample via a series of sequential dip coatings with intermediate drying and calcination at 300° C. Approximately 20 mg cobalt/gram monolith support was coated on the monolith sample with the dimensions and characteristics aforementioned. The monolith sample was crushed and sieved to approximately 1 mm size fragments and diluted with alumina.

The catalyst-coated fragments were then placed in a 6 mm cylindrical reactor tube. A catalyst prepared as described above was subjected to activation and a Fischer-Tropsch synthesis run as described in U.S. Pat. No. 7,943,674. Results are given in Table 1.

TABLE 1

| | | Comparative | |
|---|---|---|---|
| | Example 1 | Example | Example 2 |
| Time on stream | 350 | 350 | 350 |
| (TOS), h | | | |
| Monolith loading | | | |
| Catalyst, mg/g | 328 | 428 | 321 |
| Cobalt, mg/g | 25 | 86 | 21 |
| Run Conditions | | | |
| Temp, ° C. | 215 | 215 | 215 |
| Pres, atm | 20 | 20 | 20 |
| Reactant | | | |
| H₂/CO, nominal | 2.00 | 2.00 | 2.00 |
| GHSV(HFT), | 4.0 | 4.0 | 4.0 |
| SL/min | | | |
| Results | | | |
| Specific | | | |
| conversion rates | | | |
| Rate, gC/g_{Co}/h | 2.1 | 3.2 | 2.7 |
| Rate, gC/g_{Cat}/h | 0.16 | 0.63 | 0.18 |
| Rate, gC/g_{Monolith}/h | 0.05 | 0.27 | 0.06 |
| Rate, | 0.03 | 0.18 | 0.03 |
| gC/mLₒᵥₑᵣₐₗₗ/h | | | |
| Selectivities | | | |
| C₅₊, % | 50 | 64 | 54 |
| C₂₁₊, % | 1.3 | 10.7 | 1.6 |
| Branching in | 17 | 8 | 16 |
| C₅'s, % | | | |
| 1-Butene in C₄'s, % | 14 | 56 | 14 |

As can be seen from the specific conversion rates in Table 1, while conversion rates based on total monolith weight or overall volume were significantly higher for the Comparative Example (cobalt-ruthenium-manganese/alumina Fischer-Tropsch catalyst) than for Example 1 (cobalt-ruthenium/ZSM-12 integral catalyst) or Example 2 (mixed catalyst: 1 part cobalt-ruthenium-manganese/alumina Fischer-Tropsch catalyst to 2 parts HZSM-12), resulting from its higher cobalt loading, the conversion rates based on cobalt content were similar for all three types.

As can be seen from the selectivity data, the C₂₁₊ fraction produced using the Comparative Example catalyst was greater than 15% of the C₅₊ product, whereas it was only 2%-3% using the Example 1 and Example 2 catalysts. Thus, the C₅₊ liquids from the hybrid catalyst monoliths of Examples 1 and 2 were wax free at ambient temperature. The C₄ hydrocarbons from the Example hybrid catalyst monoliths had only one-fourth as much 1-butene as those from the Comparative Example, as a result of significant isomerization to internal olefins. There was also twice as much branching in the C₅ hydrocarbons from the Example hybrid catalyst monoliths compared with those from the Comparative Example. Thus, the ZSM-12 component catalyzed both double bond shifts and methyl group shifts.

The mixed catalyst of Example 2 was more active than the integral catalyst of Example 1, with similar selectivity. It was further found that mixing the small particle Fischer-Tropsch catalyst with the larger particle zeolite particles in Example 2 resulted in improved adhesion of the catalyst layer.

### Example 3

A hybrid Fischer-Tropsch catalyst of composition 7.5% Co/0.19% Ru/ZSM-12/Al₂O₃ was prepared as described in U.S. Pat. No. 7,943,674, herein incorporated by reference in its entirety. The catalyst was milled in a small colloid mill to particles having a particle size of about 25 μm. The powder was suspended in water to a solids content of 30 wt %. The pH of the suspension was about 6 for the catalysts tested.

From a commercial cordierite monolith cylinder available from Corning Inc., Corning, N.Y., were cut two samples of 3×3 cell 75 mm in length. The hybrid Fischer-Tropsch catalyst was deposited on the monolith samples via a series of sequential dip coatings with intermediate drying and calcination at 300° C. Excess liquid was removed by centrifugation. Approximately 20 mg cobalt/gram monolith support was coated on a single monolith core with the dimensions and characteristics aforementioned. The edges of the dried cores were beveled slightly to accommodate the cylindrical reactor geometry.

The catalyst-coated monolith samples were then placed in a 9.52 mm reactor tube stacking one sample on top of the other without a spacer. A catalyst prepared as described above was subjected to activation and a Fischer-Tropsch synthesis run as described in U.S. Pat. No. 7,943,674. Results are given in Table 2.

TABLE 2

| | Example 3 |
|---|---|
| Time on stream (TOS), h | 143.3 |
| Monolith loading | |
| Catalyst, mg/g | 328 |
| Cobalt, mg/g | 20 |
| Run Conditions | |
| Temp, ° C. | 215 |
| Pres, atm | 20 |
| Reactant | |
| H₂/CO, nominal | 2 |
| GHSV(HFT), SL/min | 4.9 |
| Results | |
| Specific | |
| conversion rates | |
| Rate, gC/g_{Co}/h | 1.36 |
| Rate, gC/g_{Cat}/h | 0.102 |
| Rate, gC/g_{Monolith}/h | 0.033 |
| Rate, gC/mLₒᵥₑᵣₐₗₗ/h | 0.018 |
| Selectivities | |
| CH₄, % | 18 |
| C₂, % | 2.7 |
| C₃₋₄, % | 15 |
| C₅₊, % | 63 |
| C₂₁₊, % | <1 |

It can be seen in Table 2 using the catalyst of Example 3 that a hybrid syngas conversion catalyst deposited on a monolith support can provide for a high yield of liquid hydrocarbon liquid product with low light gas without the formation of a separate solid wax phase.

Where permitted, all publications, patents and patent applications cited in this application are herein incorporated by reference in their entirety, to the extent such disclosure is not inconsistent with the present invention.

Unless otherwise specified, the recitation of a genus of elements, materials or other components, from which an individual component or mixture of components can be selected, is intended to include all possible sub-generic combinations of the listed components and mixtures thereof. Also, “comprise,” “include” and its variants, are intended to be non-limiting, such that recitation of items in a list is not to the exclusion of other like items that may also be useful in the materials, compositions, methods and systems of this invention.

From the above description, those skilled in the art will perceive improvements, changes and modifications, which are intended to be covered by the appended claims.

What is claimed is:

1. A hybrid Fischer-Tropsch monolith catalyst comprising:
a. a monolithic support; and
b. at least one catalyst layer comprising a synthesis gas conversion component and an acidic component deposited on the monolithic support.

2. The catalyst of claim 1, further comprising an interfacial layer deposited on the monolithic support between the monolithic support and the at least one catalyst layer.

3. The catalyst of claim 1, wherein the monolithic support comprises a ceramic material.

4. The catalyst of claim 1, wherein the monolithic support comprises a metallic material.

5. The catalyst of claim 1, wherein the at least one catalyst layer comprises a layer of synthesis gas conversion component deposited onto the monolithic support and a layer of acidic component deposited onto the layer of synthesis gas conversion component.

6. The catalyst of claim 1, wherein the at least one catalyst layer comprises a layer of acidic component deposited onto the monolithic support and a layer of synthesis gas conversion component deposited onto the layer of acidic component.

7. The catalyst of claim 5 or 6, wherein the layer of synthesis gas conversion component contains between about 10 and about 100 mg synthesis gas conversion component per gram of monolithic support and the layer of acidic component contains between about 10 and about 100 mg of acidic component per gram of synthesis gas conversion component.

8. The catalyst of claim 1, wherein the at least one catalyst layer comprises discrete particles of synthesis gas conversion component and discrete particles of acidic component.

9. The catalyst of claim 1, wherein the at least one catalyst layer comprises discrete integral particles comprising a synthesis gas conversion component and an acidic component.

10. The catalyst of claim 8 or 9, wherein the at least one catalyst layer contains between about 10 and about 100 mg synthesis gas conversion component per gram of monolithic support.

11. A method for forming a hybrid Fischer-Tropsch monolith catalyst comprising depositing a hybrid Fischer-Tropsch catalyst composition comprising a synthesis gas conversion component and an acidic component onto a monolithic support.

12. The method of claim 11, wherein a slurry comprising a liquid medium and the hybrid Fischer-Tropsch catalyst composition is deposited onto the monolithic support.

13. The method of claim 12, wherein the slurry contains between about 10 and about 100 mg of cobalt per gram of monolithic support.

14. The method of claim 12, wherein the slurry is deposited by wash coating, dip coating, spraying, vapor deposition or impregnation.

15. The method of claim 11, wherein the catalyst composition is deposited onto the monolithic support by first depositing a synthesis gas conversion component layer onto the monolithic support and then depositing an acidic component layer onto the synthesis gas conversion component layer.

16. The method of claim 11, wherein the catalyst composition is deposited onto the monolithic support by first depositing an acidic component layer onto the monolithic support and then depositing a synthesis gas conversion component layer onto the acidic component layer.

17. The method of claim 15 or claim 16, wherein the synthesis gas conversion component layer contains between about 10 and about 100 mg of cobalt per gram of monolithic support and the acidic component layer contains between about 10 and about 100 mg of zeolite per gram of cobalt in the synthesis gas conversion component layer.

18. The method of claim 12, wherein the slurry contains the catalyst composition in the form of integral hybrid Fischer-Tropsch catalyst particles wherein each particle comprises a synthesis gas conversion component and an acidic component.

19. The method of claim 12, wherein the slurry contains the catalyst composition in the form of discrete synthesis gas conversion component particles and discrete acidic component particles.

20. A process for synthesis gas conversion comprising:
contacting a synthesis gas feed comprising hydrogen and carbon monoxide having a H₂/CO ratio between about 1.3 and about 2.0 with the hybrid Fischer-Tropsch monolith catalyst of claim 1 in a reactor at a temperature from about 200° C. to about 260° C., a pressure from about 5 to about 30 atmospheres, and a gaseous hourly space velocity less than 20,000 volumes of gas per volume of catalyst per hour to produce a hydrocarbon product containing at least 50 wt % C₅₊ hydrocarbons.

21. A process for synthesis gas conversion comprising:
contacting a synthesis gas feed comprising hydrogen and carbon monoxide having a H₂/CO ratio between about 1.3 and about 2.0 with a Fischer-Tropsch monolith catalyst comprising a monolithic support and a catalyst layer comprising a synthesis gas conversion component deposited on the monolithic support in an alternating arrangement with a catalyst bed comprising acidic component particles in a reactor at a temperature from about 200° C. to about 260° C., a pressure from about 5 to about 40 atmospheres, and a gaseous hourly space velocity less than 20,000 volumes of gas per volume of catalyst per hour to produce a hydrocarbon product containing at least 50 wt % C₅₊ hydrocarbons.
